(19) United States
(12) Patent Application Publication
(10) Pub. No.: US 2020/0061139 A1
(43) Pub. Date: Feb. 27, 2020

(54) METHOD FOR ANTI-INFLAMMATION OF SKIN AND PROMOTING KERATINOCYTE PROLIFERATION

(71) Applicant: TCI CO., LTD, Taipei (TW)

(72) Inventor: Yung-Hsiang Lin, Taipei (TW)

(21) Appl. No.: 16/152,467

(22) Filed: Oct. 5, 2018

(30) Foreign Application Priority Data
Aug. 22, 2018 (TW) 107129335

(51) Int. Cl. A61K 36/28; A61K 8/9789; A61K 9/00; A61P 29/00; A61P 17/00

(52) U.S. Cl. CPC A61K 36/28; A61K 8/9789; A61P 17/00; A61P 29/00; A61K 9/0014

(57) ABSTRACT

Provided herein is a method for anti-inflammation of skin and promoting keratinocyte proliferation, including administering to a subject in need thereof a composition including an effective amount of an extract of Sphagneticola trilobata.

### CROSS-REFERENCE TO RELATED APPLICATION

This application claims priority of Taiwan patent application No. 107129335, filed on Aug. 22, 2018, the content of which is incorporated herein in its entirety by reference.

### BACKGROUND OF THE INVENTION

### 1. Field of the Invention

The present invention relates to a method for anti-inflammation of skin and promoting keratinocyte proliferation, comprising administering to a subject in need thereof a composition comprising an effective amount of an extract of Sphagneticola trilobata.

### 2. The Prior Art

An inflammatory response of skin refers to a series of vascular and cellular responses caused by injury or infection. Moderate skin inflammation can protect the skin from foreign substances, but excessive skin inflammation can cause redness, swelling, heat, pain and other symptoms such as allergies or redness of the skin. Existing anti-inflammatory drugs such as nonsteroidal anti-inflammatory drugs (NSAIDs) are useful for treating or alleviating many skin inflammatory reactions. However, these artificial compounds are still accompanied by varying degrees of side effects, causing trouble to the users.

In addition, the skin tissue is composed of epidermis, dermis and subcutaneous tissue. There is aging or slower keratinocyte renewal in the human skin with age, physiological factors or environmental factors. When the skin keratinocyte renewal becomes slower, the barrier function of the stratum corneum would decrease, and the skin may cause damage to the stratum corneum and produce an inflammatory reaction after being exposed to ultraviolet such as ultraviolet radiation b (UVB). Therefore, how to promote keratinocyte proliferation has become an important issue in the art.

The way commonly used to promote keratinocyte proliferation is using medicaments or skin care products applied to the skin surface. However, most of the conventional medicaments and skin care products are made of chemical components. Long-term use is not only harmful to human health, but these products are often expensive and not affordable for the users.

In order to solve the above problems, those skilled in the art urgently need to develop novel medicaments or skin care products used for anti-inflammation of skin and promoting keratinocyte proliferation for the benefit of a large group of people in need thereof.

### SUMMARY OF THE INVENTION

A primary objective of the present invention is to provide a method for anti-inflammation of skin and promoting keratinocyte proliferation, comprising administering to a subject in need thereof a composition comprising an effective amount of an extract of Sphagneticola trilobata.

According to an embodiment of the present invention, the extract of Sphagneticola trilobata is obtained by extracting Sphagneticola trilobata with water or alcohol as an extraction solvent.

According to an embodiment of the present invention, a liquid-solid ratio of the extraction solvent to the Sphagneticola trilobata is between 5-20:1-5.

According to an embodiment of the present invention, an extraction temperature ranges from 50° C. to 100° C.

According to an embodiment of the present invention, an extraction time ranges from 0.5 to 3 hours.

According to an embodiment of the present invention, the anti-inflammation of skin comprises reducing an amount of interleukin-8 (IL-8) induced by ultraviolet B (UVB).

According to an embodiment of the present invention, the composition is a pharmaceutical composition or a cosmetic composition.

According to an embodiment of the present invention, the pharmaceutical composition comprises a pharmaceutically acceptable carrier.

According to an embodiment of the present invention, the pharmaceutical composition is in a dosage form for topical administration.

According to an embodiment of the present invention, the pharmaceutical composition is in a dosage form for parenteral administration.

According to an embodiment of the present invention, the cosmetic composition is in a form for topical application.

According to an embodiment of the present invention, the cosmetic composition comprises a cosmetically acceptable adjuvant.

In summary, the extract of Sphagneticola trilobata has the effect on promoting rapid renewal and proliferation of keratinocytes, anti-inflammation of skin, and helping the skin to isolate external dirt by extracting a large amount of effective substances such as phenolic acids and diterpenoids, so that the extract of Sphagneticola trilobata enhances skin protection.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a schematic diagram showing the effect of the extract of Sphagneticola trilobata on anti-inflammation of skin.

FIG. 2 is a schematic diagram showing the effect of the extract of Sphagneticola trilobata on promoting keratinocyte proliferation.

FIG. 3 is a schematic diagram showing the effect of the extract of Sphagneticola trilobata on improving the redness of the human skin, in which “*” indicates p<0.05 when compared with week 0; “#” indicates p<0.05 when compared with the control group.

FIG. 4 is an image drawing showing the effect of the extract of Sphagneticola trilobata on improving the redness of the human skin.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

According to the present invention, Sphagneticola trilobata, English common name Yellow Dots, is a perennial herb in the family Compositae and the genus Sphagneticola with leaves opposite, thick, rough with bristles, ovate or broadly ovate, 3-lobed, serrate, and shine. The flowers are composed of lingual flowers and tubular flowers, single terminal, pedicel long, and yellow. The country of origin is from North America, and the production areas are mainly distributed in the plains and mountains of Taiwan. Because of radiant rooting and good coverage, Sphagneticola trilobata is an excellent plant for road slope protection and safety island separation. It can also be planted in gardens and flower beds for viewing. In addition, Sphagneticola trilobata can also be used as a honey plant for small butterflies and bees.

According to the present invention, the pharmaceutical composition can be manufactured to a form suitable for parenteral or topical administration, using techniques well known to those skilled in the art, including, but not limited to, injection (e.g., sterile aqueous solution or dispersion), sterile powder, external preparation, and the like.

According to the present invention, the pharmaceutical composition may further comprise a pharmaceutically acceptable carrier which is widely used in pharmaceutically manufacturing techniques. For example, the pharmaceutically acceptable carrier can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), aqueous solution containing alcohol, and combinations thereof.

According to the present invention, the pharmaceutical composition can be administered by parenteral routes selected from the group consisting of subcutaneous injection, intraepidermal injection, intradermal injection, intradermal injection, and intralesional injection.

According to the present invention, the pharmaceutical composition can be manufactured to an external preparation suitable for topical application to the skin using techniques well known to those skilled in the art, including, but not limited to, emulsion, coagulation, gel, ointment, cream, patch, liniment, powders, aerosol, spray, lotion, serum, paste, foam, drop, suspension, salve, and bandage.

According to the present invention, the external preparation is prepared by mixing the pharmaceutical composition of the present invention with a base well known to those skilled in the art.

According to the present invention, the base may comprise one or more additives selected from the group consisting of water, alcohols, glycol, hydrocarbons such as petroleum jelly and white petrolatum, wax such as paraffin and yellow wax, preserving agents, antioxidants, surfactants, absorption enhancers, stabilizing agents, gelling agents such as Carbopol® 974P, microcrystalline cellulose and carboxymethylcellulose, active agents, humectants, odor absorbers, fragrances, pH adjusting agents, chelating agents, emulsifiers, occlusive agents, emollients, thickeners, solubilizing agents, penetration enhancers, anti-irritants, colorants, and propellants. The selection and quantity of these additives fall within the scope of professional literacy and routine techniques of those skilled in the art.

According to the present invention, the cosmetic composition can further comprise an acceptable adjuvant that is widely used in the manufacture of cosmetic compositions. For example, the acceptable adjuvant may comprise one or more reagents selected from the group consisting of solvents, gelling agents, active agents, preservatives, antioxidants, screening agents, chelating agents, surfactants, coloring agents, thickening agents, fillers, fragrances, and odor absorbers. The selection and quantity of these reagents fall within the scope of professional literacy and routine techniques of those skilled in the art.

According to the present invention, the cosmetic composition can be manufactured to a form suitable for skincare or makeup using techniques well known to those skilled in the art, including, but not limited to, aqueous solution, aqueous-alcohol solution or oily solution, oil-in-water type, water-in-oil type or composite type emulsion, gel, ointment, cream, mask, patch, pack, liniment, powders, aerosol, spray, lotion, serum, paste, foam, dispersion, drop, mousse, sunblock, tonic water, foundation, makeup remover products, soap, and other body cleansing products.

According to the present invention, the cosmetic composition may also be used in combination with one or more external use agents with known activities selected from the group consisting of whitening agents such as tretinoin, catechin, kojic acid, arbutin and vitamin C, humectants, anti-inflammatory agents, bactericides, ultraviolet absorbers, plant extracts extracts such as aloe extract, skin nutrients, anesthetics, anti-acne agents, antipruritics, analgesics, antidermatitis agents, antihyperkeratolytic agents, anti-dry skin agents, antipsoriatic agents, antiaging agents, antiwrinkle agents, antiseborrheic agents, wound-healing agents, corticosteroids, and hormones. The selection and quantity of these external use agents fall within the scope of professional literacy and routine techniques of those skilled in the art.

### Example 1

Preparation of Extract of Sphagneticola trilobata

First, the whole plant of Sphagneticola trilobata (obtained from farmers) was washed for subsequent extraction. The whole plant of washed Sphagneticola trilobata was homogenized, and the homogenate was extracted with water or alcohols as an extraction solvent. In an embodiment of the present invention, the extraction solvent is water, and a liquid-solid ratio of the extraction solvent to the Sphagneticola trilobata is between 5-20:1-5. The extraction was performed under 50° C. to 100° C. for 0.5 to 3 hours. Thereafter, the obtained mixture was cooled to room temperature and filtered through a 400-mesh strainer, and the filtered product was concentrated under reduced pressure at 45° C. to 70° C. to obtain the extract of Sphagneticola trilobata.

### Example 2

Evaluation of Effect of Extract of Sphagneticola trilobata on Anti-Inflammation of Skin

Interleukin-8 (IL-8) is a known cytokine, and it has a function of promoting the production of an inflammatory reaction. When an inflammatory reaction occurs, inflammatory tissues release specific cytokines (such as IL-1, IL-6 and IL-8) to attract specific functional cells to specific tissues, wherein IL-8 plays an important role in the chemotaxis of neutrophils. First, human primary epidermal keratinocytes (HPEKp) were subjected to the experiment of keratinocyte proliferation. The human primary epidermal keratinocytes were purchased from CELLnTEC (Switzerland) under the number HPEK-50. The cells were cultured in serum-free keratinocyte-SFM (Gibco, USA, #10724-011). 500 μL of the medium was added to each well of a 24-well culture plate to have 5×10⁴ cells per well. After 24 hours of incubation at 37° C., the medium was removed.

Thereafter, four groups of human primary epidermal keratinocytes (i.e., a control group, a UVB group and two experimental groups referred to as Experimental Group 1 and Experimental Group 2) were prepared. The UVB group, the Experimental Group 1 and the Experimental Group 2 were irradiated with UVB at a dose of 300 mJ/cm² using a UV irradiation chamber (Vilber) in order to induce inflammatory reactions, and 0.03125 mg/mL and 0.0625 mg/mL extract of Sphagneticola trilobata were added to the cells in the Experimental Group 1 and the Experimental Group 2, respectively. The cells in the control group were left untreated. After the cell cultures in each group were cultured at 37° C. for 24 hours, 120 μL of the cell culture supernatant was used as a sample for analysis.

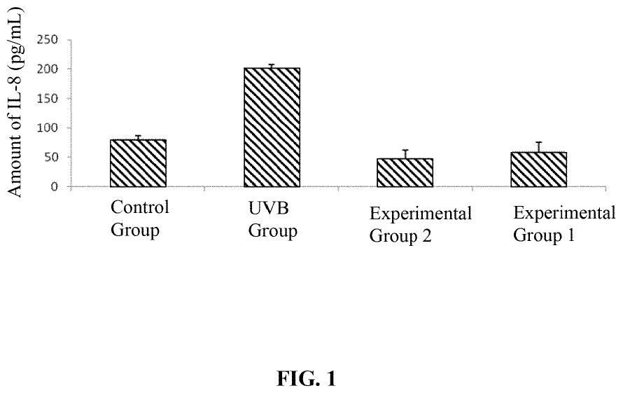

Subsequently, the human CXCL8/IL8 ELISA kit (purchased from R&D systems, USA) was used to detect the amount of interleukin-8 (IL-8) produced by each group. The capture antibody was diluted with PBS, and the diluted capture antibody was coated to the bottom of a 96-well microplate in a volume of 100 μL per well, followed by reaction at 4° C. overnight. The 96-well microplate was washed 3 times with 200 μL of wash buffer (containing 0.05% Tween 20 in PBS), and blocking was carried out using 300 μL of Block buffer (1% BSA in PBS), followed by reaction at 37° C. for 3 hours. Thereafter, 100 μL of the sample and the standard mixed with a reagent Diluent (0.1% BSA in PBS) were added, followed by binding to the capture antibody at 37° C. for 2 hours. The 96-well microplate was washed 3 times with 200 μL of wash buffer, and 100 μL of the detection antibody was added, followed by detecting the capture antibody at 37° C. for 2 hours. Subsequently, the 96-well microplate was washed 3 times with 200 μL of wash buffer, and 100 μL of Streptavidin-HRP was added, followed by treatment at room temperature for 20 minutes. The 96-well microplate was washed 3 times with 200 μL of wash buffer, and 100 μL of a substrate solution (R&D systems) was added, followed by treatment at room temperature for 20 minutes. 50 μL of a stop solution was added to stop the reaction, and the absorbance at 450 nm was measured using an ELISA reader. Statistical analysis was performed using Excel. Statistically significant differences between each group were determined by the Student's t-test. The result of Example 2 is shown in FIG. 1.

FIG. 1 is a schematic diagram showing the effect of the extract of Sphagneticola trilobata on anti-inflammation of skin. As shown in FIG. 1, the amount of IL-8 produced by the UVB group is significantly increased compared with those of the control group, indicating that UVB produces an inflammatory response to human primary epidermal keratinocytes. Compared with the UVB group, the amount of IL-8 produced by the Experimental Group 1 is decreased by 70.17%, the amount of IL-8 produced by the Experimental Group 2 is decreased by 75.65%, and there is a significant decrease with the increase of the concentration of the extract of Sphagneticola trilobata. The result of this example indicates that the extract of Sphagneticola trilobata has the effect on anti-inflammation of skin and can enhance skin protection.

### Example 3

Evaluation of Effect of Extract of Sphagneticola trilobata on Promoting Keratinocyte Proliferation

First, human primary epidermal keratinocytes were cultured in serum-free keratinocyte-SFM (Gibco, USA, #10724-011). The medium was added to each well of a 96-well culture plate to have 3,000 cells per well, followed by incubation at 37° C. for 2 hours.

Thereafter, three groups of human primary epidermal keratinocytes (i.e., a control group and two experimental groups referred to as Experimental Group 1 and Experimental Group 2) were prepared. 10 μL of 100 μM BrdU labeling reagent (Roche; 11647229001) was added to each group, and 0.03125 mg/mL and 0.0625 mg/mL extract of Sphagneticola trilobata were added to the cells in the Experimental Group 1 and the Experimental Group 2, respectively, followed by incubation for 24 hours. The supernatant was removed and 200 μL of FixDenat was added to each well, followed by reaction at room temperature for 30 minutes. Subsequently, the FixDenat solution was removed and the wells were washed with 1×PBS once, and the anti-BrdU-POD working solution (anti-BrdU-POD and antibody dilution solution with a diluted ratio of 1:100) was added, followed by reaction at room temperature for 90 minutes. The antibody conjugate was removed and the residue was rinsed well 3 times with 200-300 μL of washing solution. The washing solution was removed and 100 μL of a substrate solution (i.e., tetramethyl-benzidine, TMB) was added to each well to color, followed by standing at room temperature for 5 to 30 minutes. Thereafter, 25 μL of 1M H₂SO₄ was added to each well, followed by reaction on a shaker at 300 rpm for approximately 1 minute. The absorbance at 450 nm was measured using an ELISA reader (BioTek, USA). Statistical analysis was performed using Excel.

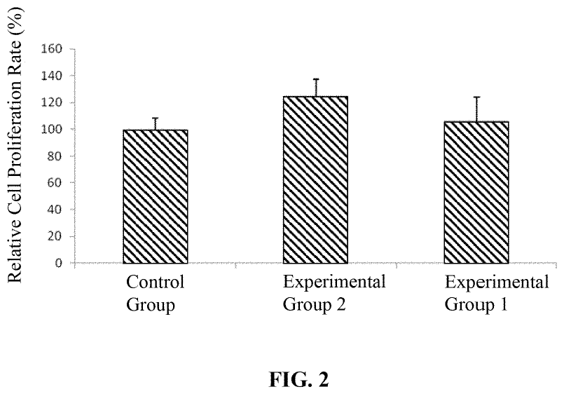

Statistically significant differences between each group were determined using Excel. The result of Example 3 is shown in FIG. 2.

FIG. 2 is a schematic diagram showing the effect of the extract of Sphagneticola trilobata on promoting keratinocyte proliferation. As shown in FIG. 2, compared with the control group, the relative cell proliferation rate of the Experimental Group 1 is increased by 6.4%, the relative cell proliferation rate of the Experimental Group 2 is increased by 24.9%, and there is a significant increase with the increase of the concentration of the extract of Sphagneticola trilobata. The result of this example indicates that the extract of Sphagneticola trilobata has the effect on promoting keratinocyte proliferation, thereby promoting rapid renewal of keratinocytes and helping the skin to isolate external dirt.

### Example 4

Human Body Function Test of Extract of Sphagneticola trilobata

In the present example, the essence comprising 2% of the extract of Sphagneticola trilobata (hereinafter referred to as “Sphagneticola trilobata essence”) was used to detect whether it has an effect on improving redness of the human skin.

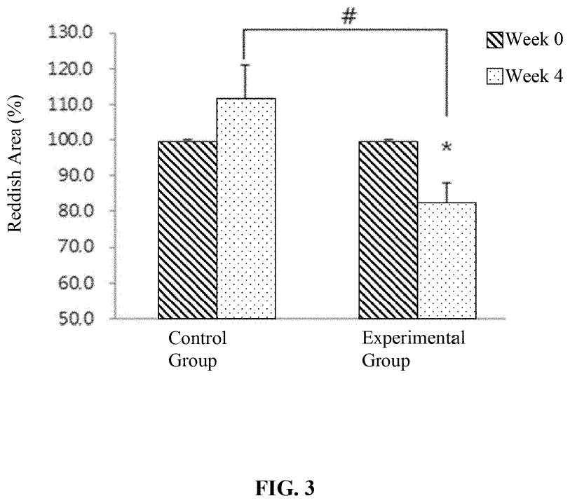

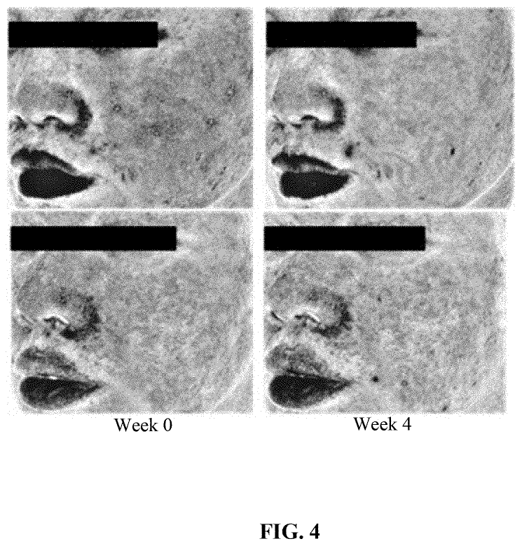

First, 8 subjects were recruited, the right face of each subject was used as a control group, and the left face was used as an experimental group. After cleaning the face every morning and evening, the placebo was applied to the skin of the control group, and the Sphagneticola trilobata essence was applied to the skin of the experimental group. The massage was promoted by a slight massage on the fingertips, and the skin redness was detected before use (week 0) and at the fourth week after use (week 4). The VISIA full-face skin detector was used to analyze the reddish value of the whole face skin using multiple spectral imaging techniques. The red (dark) area of the skin is a condition that detects the inflammation and sensitivity of the face. The lower the value represents the lower the level of sensitivity and inflammation. The results of Example 4 are shown in FIG. 3 and FIG. 4.

FIG. 3 is a schematic diagram showing the effect of the extract of Sphagneticola trilobata on improving the redness of the human skin. FIG. 4 is an image drawing showing the effect of the extract of Sphagneticola trilobata on improving the redness of the human skin. As shown in FIG. 3, compared with week 0, the reddish area (%) of the experimental group is significantly reduced (by 17.5%) at week 4 after use; compared with the control group, the reddish area (%) is significantly reduced. As shown in FIG. 4, compared with week 0, the reddish area of the experimental group is significantly reduced at week 4 after use, and the subject improvement ratio is 87.5%. The result of this example indicates that the extract of Sphagneticola trilobata has the effect on improving redness of the human skin.

In summary, the extract of Sphagneticola trilobata has the effect on promoting rapid renewal and proliferation of keratinocytes, anti-inflammation of skin (e.g., improving skin redness), and helping the skin to isolate external dirt by extracting a large amount of effective substances, so that the extract of Sphagneticola trilobata enhances skin protection.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for anti-inflammation of skin and promoting keratinocyte proliferation, comprising administering to a subject in need thereof a composition comprising an effective amount of an extract of Sphagneticola trilobata.

2. The method according to claim 1, wherein the extract of Sphagneticola trilobata is obtained by extracting Sphagneticola trilobata with water or alcohol as an extraction solvent.

3. The method according to claim 2, wherein a liquid-solid ratio of the extraction solvent to the Sphagneticola trilobata is between 5-20:1-5.

4. The method according to claim 2, wherein an extraction temperature ranges from 50° C. to 100° C.

5. The method according to claim 2, wherein an extraction time ranges from 0.5 to 3 hours.

6. The method according to claim 1, wherein the anti-inflammation of skin comprises reducing an amount of interleukin-8 (IL-8) induced by ultraviolet B (UVB).

7. The method according to claim 1, wherein the composition is a pharmaceutical composition or a cosmetic composition.

8. The method according to claim 7, wherein the pharmaceutical composition comprises a pharmaceutically acceptable carrier.

9. The method according to claim 7, wherein the pharmaceutical composition is in a dosage form for topical administration.

10. The method according to claim 7, wherein the pharmaceutical composition is in a dosage form for parenteral administration.

11. The method according to claim 7, wherein the cosmetic composition is in a form for topical application.

12. The method according to claim 7, wherein the cosmetic composition comprises a cosmetically acceptable adjuvant.
